SEQUENCE LISTING (21 sequences)

SEQ ID NO: 1 - protein, 131 aa, Pan troglodytes
EVQLLESGPGLVKPSQTLSLTCAVSGAFMTSDYLYWSWLRQPPGKGPEWIGGIYTTGRSHYNPSLRGRATLSVDTSKNQFSLELNSVTAADTAVYYCARGQYHNWGPSLFDYYNYYYMDVWGKGTTVTVSS

SEQ ID NO: 2 - protein, 31 aa, Pan troglodytes
EVQLLESGPGLVKPSQTLSLTCAVSGAFMTS

SEQ ID NO: 3 - protein, 6 aa, Pan troglodytes
DYLYWS

SEQ ID NO: 4 - protein, 14 aa, Pan troglodytes
WLRQPPGKGPEWIG

SEQ ID NO: 5 - protein, 16 aa, Pan troglodytes
GIYTTGRSHYNPSLRG

SEQ ID NO: 6 - protein, 32 aa, Pan troglodytes
RATLSVDTSKNQFSLELNSVTAADTAVYYCAR

SEQ ID NO: 7 - protein, 21 aa, Pan troglodytes
GQYHNWGPSLFDYYNYYYMDV

SEQ ID NO: 8 - protein, 11 aa, Pan troglodytes
WGKGTTVTVSS

SEQ ID NO: 9 - protein, 109 aa, Pan troglodytes
AAELTQSPSSLSASVGDRVTITCRASQDISNYLAWYQQKPGKAPKLLIYSASRLQSGVPSRFSGSGSGTDYTLTLSSLHPEDSATYYCHQYHSAPWTFGQGTKVEIKRT

SEQ ID NO: 10 - protein, 23 aa, Pan troglodytes
AAELTQSPSSLSASVGDRVTITC

SEQ ID NO: 11 - protein, 11 aa, Pan troglodytes
RASQDISNYLA

SEQ ID NO: 12 - protein, 15 aa, Pan troglodytes
WYQQKPGKAPKLLIY

SEQ ID NO: 13 - protein, 7 aa, Pan troglodytes
SASRLQS

SEQ ID NO: 14 - protein, 32 aa, Pan troglodytes
GVPSRFSGSGSGTDYTLTLSSLHPEDSATYYC

SEQ ID NO: 15 - protein, 9 aa, Pan troglodytes
HQYHSAPWT

SEQ ID NO: 16 - protein, 12 aa, Pan troglodytes
FGQGTKVEIKRT

SEQ ID NO: 17 - DNA, 30 bp, Artificial Sequence
  synthetic primer
gcatgtactagttgtgtcacaagatttggg

SEQ ID NO: 18 - DNA, 21 bp, Artificial Sequence
  synthetic primer
attgcctacggcagccgctgg

SEQ ID NO: 19 - DNA, 21 bp, Artificial Sequence
  synthetic primer
ggaagtagtccttgaccaggc

SEQ ID NO: 20 - DNA, 21 bp, Artificial Sequence
  synthetic primer
acagctatcgcgattgcagtg

SEQ ID NO: 21 - DNA, 21 bp, Artificial Sequence
  synthetic primer
cacctgatcctcagatggcgg

(19) United States
(12) Patent Application Publication
(10) Pub. No.: US 2011/0097270 A1
(43) Pub. Date: Apr. 28, 2011

(54) Monoclonal Antibodies That Bind or Neutralize Hepatitis B Virus

(76) Inventors: Darren J. Schofield, Hertfordshire (GB); Suzanne U. Emerson, Gaithersburg, MD (US); Robert H. Purcell, Gaithersburg, MD (US)

(21) Appl. No.: 11/795,255

(22) PCT Filed: Jan. 13, 2006
(86) PCT No.: PCT/US2006/001336

§ 371 Date: Oct. 29, 2009

Related U.S. Application Data
(60) Provisional application No. 60/644,309, filed on Jan. 14, 2005.

(51) Int. Cl. A61K 49/00; C07K 16/08; C07H 21/00; C12N 15/63; C12N 5/10; A61K 39/395; C12Q 1/70; A61P 31/20; A61P 37/04

(52) U.S. Cl. 424/9.1; 530/387.3; 536/23.53; 435/320.1; 435/325; 424/133.1; 435/5

(57) ABSTRACT

The present invention relates to the isolation and characterization of a novel neutralizing chimpanzee monoclonal antibody to hepatitis B virus. The invention provides such antibodies, fragments of such antibodies retaining hepatitis B virus-binding ability, fully human or humanized antibodies retaining hepatitis B virus-binding ability, and pharmaceutical compositions including such antibodies. The invention further provides for isolated nucleic acids encoding the antibodies of the invention and host cells transformed therewith. Additionally, the invention provides for prophylactic, therapeutic, and diagnostic methods employing the antibodies and nucleic acids of the invention.

### RELATED APPLICATIONS

This application claims the benefit of a U.S. Provisional Patent application No.: 60/644,309, filed Jan. 14, 2005.

### FIELD OF THE INVENTION

This invention relates generally to the field of immunology and specifically to monoclonal antibodies that bind or neutralize hepatitis B virus.

### BACKGROUND OF THE INVENTION

Hepatitis B virus (HBV) causes acute resolving hepatitis, fulminant hepatitis and chronic infection in humans. Most people infected with HBV develop an acute hepatitis, from which complete recovery is usual. Viral DNA disappears from circulation and serum antibodies to hepatitis B surface antigen (HBsAg) appear during convalescence. These antibodies (anti-HBs) protect against re-infection with HBV. However, there are estimated to be more than 250 million people worldwide who are chronic carriers of HBV, who fail to produce anti-HBs. Furthermore, a significant proportion of these chronically infected individuals will develop either liver cirrhosis or hepatocellular carcinoma in later life. The commercially available recombinant HBsAg vaccines generate protective or neutralizing antibodies, and have reduced the need to give anti-HBs immunoglobulins to protect from HBV infections. However, instances remain when anti-HBV immune globulins are still administered, usually in conjunction with vaccination, e.g., needlestick injury with HBV contaminated material, and perinatal exposure of infants to their HBV positive mothers. In addition, liver transplant recipients who are chronically infected with HBV receive anti-HBV immune globulin in an attempt to prevent recurrence of HBV replication in the transplanted liver. Therefore, there is a need for the generation of high affinity, neutralizing human monoclonal antibodies (MAbs) as an approach to immunoprophylaxis and immunotherapy of HBV infections.

### SEGUE TO THE INVENTION

Bacteriophage (phage) particles displaying libraries of antibody fragments on their surface have provided a powerful tool for the generation of human monoclonal antibodies (MAbs) to a variety of infectious agents (e.g., human immunodeficiency virus type 1 (HIV-1), hepatitis C virus, Ebola virus) as well as to cancer markers (e.g., melanoma, adenocarcinoma, ovarian carcinoma). MAbs produced from human antibody gene libraries have the potential to serve directly as immune prophylactic reagents against infectious agents, when vaccines are not commercially available. Typically, cDNA antibody libraries are derived from patients infected with one particular virus, or have one particular type of cancer. Herein we describe the use of the chimpanzee (Pan troglodytes) as an alternative donor for library construction. A combinatorial antibody library was generated from a chimpanzee that had been sequentially infected with the five recognized hepatitis-causing viruses, hepatitis A, B, C, D and E viruses. Unlike most humans, the chimpanzee was seropositive for antibodies to all five hepatitis viruses. Herein we describe the generation of a neutralizing MAb to one of these viruses, HBV, and its characterization as an antibody directed against the principle neutralizing epitope (a) of HBsAg.

### SUMMARY OF THE INVENTION

The present invention relates to the isolation and characterization of a novel neutralizing chimpanzee monoclonal antibody to hepatitis B virus. The invention provides such antibodies, fragments of such antibodies retaining hepatitis B virus-binding ability, fully human or humanized antibodies retaining hepatitis B virus-binding ability, and pharmaceutical compositions including such antibodies. The invention further provides for isolated nucleic acids encoding the antibodies of the invention and host cells transformed therewith. Additionally, the invention provides for prophylactic, therapeutic, and diagnostic methods employing the antibodies and nucleic acids of the invention.

### BRIEF DESCRIPTION OF THE DRAWINGS

FIG. 1. The deduced amino acid sequence of the HBV#8 γ1-heavy chain and κ-light chain (FR, framework region; CDR, complementarity determining region).

Brief Description of the SEQ ID NOs.

| | Heavy Chain | Light Chain |
|---|---|---|
| | HBV#8 Sequence | HBV#8 Sequence |
| Region | SEQ ID NO: 1 | SEQ ID NO: 9 |
| FR1 | SEQ ID NO: 2 | SEQ ID NO: 10 |
| CDR1 | SEQ ID NO: 3 | SEQ ID NO: 11 |
| FR2 | SEQ ID NO: 4 | SEQ ID NO: 12 |
| CDR2 | SEQ ID NO: 5 | SEQ ID NO: 13 |
| FR3 | SEQ ID NO: 6 | SEQ ID NO: 14 |
| CDR3 | SEQ ID NO: 7 | SEQ ID NO: 15 |
| FR4 | SEQ ID NO: 8 | SEQ ID NO: 16 |

### DEPOSIT OF BIOLOGICAL MATERIAL

The following biological material has been deposited in accordance with the terms of the Budapest Treaty with the American Type Culture Collection (ATCC), Manassas, Va., on the date indicated:

| | Designation | |
|---|---|---|
| Biological material | No. | Date |
| Plasmid of Fab HBV#8 derived from | PTA-6098 | Jun. 22, 2004 |
| E. coil XL-1Blue: pCOMB3H/6 HIS-HBV#8 | | |

Plasmid of Fab HBV#8 derived from E. coli XL-1Blue: pCOMB3H/6 HIS-HBV#8 was deposited as ATCC Accession No. PTA-6098 on Jun. 22, 2004 with the American Type Culture Collection (ATCC), 10801 University Blvd., Manassas, Va. 20110-2209, USA. This deposit was made under the provisions of the Budapest Treaty on the International Recognition of the Deposit of Microorganisms for the Purposes of Patent Procedure and the Regulations thereunder (Budapest Treaty). This assures maintenance of a viable culture of the deposit for 30 years from date of deposit. The deposit will be made available by ATCC under the terms of the Budapest Treaty, and subject to an agreement between Applicant and ATCC which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC §122 and the Commissioner's rules pursuant thereto (including 37 CFR §1.14). Availability of the deposited biological material is not to be construed as a license to practice the invention in contravention of the rights granted under the authority of any government in accordance with its patent laws.

### DETAILED DESCRIPTION OF THE PREFERRED EMBODIMENT

A combinatorial antibody library of γ1-heavy and κ-light chain genes was constructed from bone marrow cells of a chimpanzee experimentally infected with hepatitis B virus (HBV). The antibody library was displayed on the surface of bacteriophage particles. Antibodies to HBV were selected by panning on hepatitis B surface antigen (HBsAg). One HBsAg-specific Fab, HBV#8 was isolated. The affinity (K_{d}) of HBV#8 Fab for HBsAg was 1.9×10⁻⁶M. Conversion of the Fab to a whole IgG molecule improved the affinity by 75-fold, to 2.5×10⁻⁸M. Despite being of relatively low affinity, HBV#8 Fab neutralized HBV in a primary hepatocyte cell culture system: the level of HBsAg recovered from the infected cell supernatant was reduced by >90% compared to the control. Also, HBV DNA was diminished in cell lysates. Competition studies showed that this MAb did not bind to previously mapped neutralization epitopes in the proposed first or second loops of the HBsAg a determinant.

### Definitions

As used herein, the term “antibody” means an immunoglobulin molecule or a fragment of an immunoglobulin molecule having the ability to specifically bind to a particular antigen. Antibodies are well known to those of ordinary skill in the science of immunology. As used herein, the term “antibody” means not only full-length antibody molecules but also fragments of antibody molecules retaining antigen binding ability. Such fragments are also well known in the art and are regularly employed both in vitro and in vivo. In particular, as used herein, the term “antibody” means not only full-length immunoglobulin molecules but also antigen binding active fragments such as the well-known active fragments F(ab′)₂, Fab, Fv, and Fd.

As used herein, the term “HBV disease” means any disease caused, directly or indirectly, by a hepatitis B virus (HBV). HBV is associated with a wide spectrum of liver disease, from a subclinical carrier state to acute hepatitis, chronic hepatitis, cirrhosis, and hepatocellular carcinoma. It also has an association with several primarily nonhepatic disorders including polyarteritis nodosa and other collagen vascular diseases, membranous glomerulonephritis, essential mixed cryoglobulinemia, and popular acrodermatitis of childhood.

As used herein with respect to polypeptides, the term “substantially pure” means that the polypeptides are essentially free of other substances with which they may be found in nature or in vivo systems to an extent practical and appropriate for their intended use. In particular, the polypeptides are sufficiently pure and are sufficiently free from other biological constituents of their hosts cells so as to be useful in, for example, generating antibodies, sequencing, or producing pharmaceutical preparations. By techniques well known in the art, substantially pure polypeptides may be produced in light of the nucleic acid and amino acid sequences disclosed herein. Because a substantially purified polypeptide of the invention may be admixed with a pharmaceutically acceptable carrier in a pharmaceutical preparation, the polypeptide may comprise only a certain percentage by weight of the preparation. The polypeptide is nonetheless substantially pure in that it has been substantially separated from the substances with which it may be associated in living systems.

As used herein with respect to nucleic acids, the term “isolated” means: (1) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

As used herein, a coding sequence and regulatory sequences are said to be “operably joined” when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribing and 5′ non-translating sequences involved with initiation of transcription and translation respectively, such as a TATA box, capping sequence, CAAT sequence, and the like. Especially, such 5′ non-transcribing regulatory sequences will include a promoter region which includes a promoter sequence for transcriptional control of the operably joined gene. Regulatory sequences may also include enhancer sequences or upstream activator sequences, as desired.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification and selection of cells which have been transformed or transfected with the vector. Markers include, for example, genes encoding proteins which increase or decrease either resistance or sensitivity to antibiotics or other compounds, genes which encode enzymes whose activities are detectable by standard assays known in the art (e.g., B-galactosidase or alkaline phosphatase), and genes which visibly affect the phenotype of transformed or transfected cells, hosts, colonies or plaques. Preferred vectors are those capable of autonomous replication and expression of the structural gene products present in the DNA segments to which they are operably joined.

### Novel Anti-HBV Monoclonal Antibodies

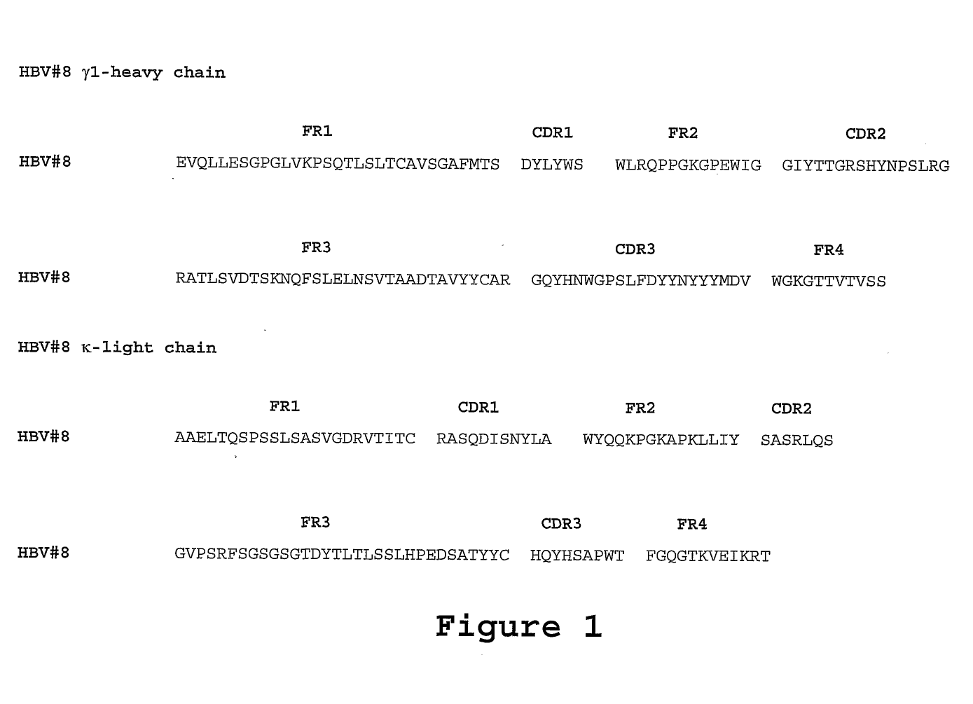

The present invention derives, in part, from the isolation and characterization of a novel chimpanzee monoclonal antibody that selectively binds and neutralizes HBV and that we have designated HBV#8. The paratope of the HBV#8 monoclonal antibody associated with the neutralization epitope on the HBV is defined by the amino acid (aa) sequences of the immunoglobulin heavy and light chain V-regions depicted in FIG. 1 and SEQ ID NO: 1 and SEQ ID NO: 9. The nucleic acid sequences coding for these aa sequences were identified by sequencing the Mab heavy chain and light chain fragments. Due to the degeneracy of the DNA code, the paratope is more properly defined by the derived aa sequences depicted in FIG. 1 and SEQ ID NO: 1 and SEQ ID NO: 9.

In one set of embodiments, the present invention provides the full-length, humanized monoclonal antibody of the HBV#8 antibody, or other HBV antibody in isolated form and in pharmaceutical preparations. Similarly, as described herein, the present invention provides isolated nucleic acids, host cells transformed with nucleic acids, and pharmaceutical preparations including isolated nucleic acids, encoding the full-length, humanized monoclonal antibody of the HBV#8 antibody, or other HBV antibody. Finally, the present invention provides methods, as described more fully herein, employing these antibodies and nucleic acids in the in vitro and in vivo diagnosis, prevention and therapy of HBV disease.

Significantly, as is well-known in the art, only a small portion of an antibody molecule, the paratope, is involved in the binding of the antibody to its epitope (see, in general, Clark, W. R. (1986) The Experimental Foundations of Modem Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of a full-length antibody. Similarly, an antibody from which the Fc region has been enzymatically cleaved, or which has been produced without the Fc region, designated an Fab fragment, retains one of the antigen binding sites of a full-length antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986, supra; Roitt, 1991, supra). In both the heavy chain Fd fragment and the light chain of IgG immunoglobulins, there are four framework regions (FRI through FR4) separated respectively by three complementarity determining ‘regions (CDR1 through CDR3). The CDRs, and in particular the CDR3 regions, and more particularly the heavy chain CDR3, are largely responsible for antibody specificity.

The complete amino acid sequences of the antigen-binding Fab portion of the of the HBV#8 monoclonal antibody as well as the relevant FR and CDR regions are disclosed herein. SEQ ID NO: 1 discloses the amino acid sequence of the Fd fragment of HBV#8. The amino acid sequences of the heavy chain FR1, CDR1, FR2, CDR2, FR3, CDR3 and FR4 regions are disclosed as SEQ ID NO: 2 through SEQ ID NO: 8, respectively. SEQ ID NO: 9 discloses the amino acid sequence of the light chain of HBV#8. The amino acid sequences of the light chain FR1, CDR1, FR2, CDR2, FR3, CDR3 and FR4 regions are disclosed as SEQ ID NO: 10 through SEQ ID NO: 16, respectively.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. Thus, for example, PCT International Publication Number WO 92/04381 teaches the production and use of humanized murine RSV antibodies in which at least a portion of the murine FR regions have been replaced by FR regions of human origin. Such antibodies, including fragments of full-length antibodies with antigen-binding ability, are often referred to as “chimeric” antibodies.

Thus, as will be apparent to one of ordinary skill in the art, the present invention also provides for F(ab′)₂, Fab, Fv and Fd fragments of the HBV#8 antibody, or other HBV antibody; chimeric antibodies in which the Fc and/or FR and/or CDR1 and/or CDR2 and/or light chain CDR3 regions of the HBV#8 antibody, or other HBV antibody, have been replaced by homologous human or non-human sequences; chimeric F(ab′)₂ fragment antibodies in which the FR and/or CDR1 and/or CDR2 and/or light chain CDR3 regions of the HBV#8 antibody, or other HBV antibody, have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. Thus, those skilled in the art may alter the HBV#8 antibody, or other HBV antibody, by the construction of CDR grafted or chimeric antibodies or antibody fragments containing all, or part thereof, of the disclosed heavy and light chain V-region CDR aa sequences (Jones, P. T. et al. 1986 Nature 321:522; Verhoeyen, M. et al. 1988 Science 39:1534; and Tempest, P. R. et al. 1991 Bio/Technology 9:266), without destroying the specificity of the antibodies for the HBV epitope. Such CDR grafted or chimeric antibodies or antibody fragments can be effective in prevention and treatment of HBV infection in humans.

In preferred embodiments, the chimeric antibodies of the invention are fully human or humanized chimpanzee monoclonal antibodies including at least the heavy chain CDR3 region of the HBV#8 antibody, or other HBV antibody. As noted above, such chimeric antibodies may be produced in which some or all of the FR regions of the HBV#8 antibody, or other HBV antibody, have been replaced by other homologous human FR regions. In addition, the Fc portions may be replaced so as to produce IgA or IgM as well as IgG antibodies bearing some or all of the CDRs of the HBV#8 antibody, or other HBV antibody. Of particular importance is the inclusion of the heavy chain CDR3 region and, to a lesser extent, the other CDRs of the HBV#8 antibody, or other HBV antibody. Such fully human or humanized chimpanzee monoclonal antibodies will have particular utility in that they will not evoke an immune response against the antibody itself in humans.

It is also possible, in accordance with the present invention, to produce chimeric antibodies including non-human sequences. Thus, one may use, for example, murine, ovine, equine, bovine or other mammalian Fc or FR sequences to replace some or all of the Fc or FR regions of the HBV#8 antibody, or other HBV antibody. Some of the CDRs may be replaced as well. Again, however, it is preferred that at least the heavy chain CDR3 of the HBV#8 antibody, or other HBV antibody, be included in such chimeric antibodies and, to a lesser extent, it is also preferred that some or all of the other CDRs of the HBV#8 antibody, or other HBV antibody, be included. Such chimeric antibodies bearing non-human immunoglobulin sequences admixed with the CDRs of the HBV#8 antibody, or other HBV antibody, are not preferred for use in humans and are particularly not preferred for extended use because they may evoke an immune response against the non-human sequences. They may, of course, be used for brief periods or in immunosuppressed individuals but, again, fully human or humanized chimpanzee monoclonal antibodies are preferred. Because such antibodies may be used for brief periods or in immunosuppressed subjects, chimeric antibodies bearing non-human mammalian Fc and FR sequences but including at least the heavy chain CDR3 of the HBV#8 antibody, or other HBV antibody, are contemplated as alternative embodiments of the present invention.

For inoculation or prophylactic uses, the antibodies of the present invention are preferably full-length antibody molecules including the Fc region. Such full-length antibodies will have longer half-lives than smaller fragment antibodies (e.g., Fab) and are more suitable for intravenous, intraperitoneal, intramuscular, intracavity, subcutaneous, or transdermal administration.

In some embodiments, Fab fragments, including chimeric Fab fragments, are preferred. Fabs offer several advantages over F(ab′)₂ and whole immunoglobulin molecules for this therapeutic modality. First, because Fabs have only one binding site for their cognate antigen, the formation of immune complexes is precluded whereas such complexes can be generated when bivalent F(ab′)₂ s and whole immunoglobulin molecules encounter their target antigen. This is of some importance because immune complex deposition in tissues can produce adverse inflammatory reactions. Second, because Fabs lack an Fc region they cannot trigger adverse inflammatory reactions that are activated by Fc, such as activation of the complement cascade. Third, the tissue penetration of the small Fab molecule is likely to be much better than that of the larger whole antibody. Fourth, Fabs can be produced easily and inexpensively in bacteria, such as E. coli, whereas whole immunoglobulin antibody molecules require mammalian cells for their production in useful amounts. The latter entails transfection of immunoglobulin sequences into mammalian cells with resultant transformation. Amplification of these sequences must then be achieved by rigorous selective procedures and stable transformants must be identified and maintained. The whole immunoglobulin molecules must be produced by stably transformed, high expression mammalian cells in culture with the attendant problems of serum-containing culture medium. In contrast, production of Fabs in E. coli eliminates these difficulties and makes it possible to produce these antibody fragments in large fermenters which are less expensive than cell culture-systems.

In addition to Fabs, smaller antibody fragments and epitope-binding peptides having binding specificity for the epitope defined by the HBV#8 antibody, or other HBV antibody, are also contemplated by the present invention and can also be used to bind or neutralize the virus. For example, single chain antibodies can be constructed according to the method of U.S. Pat. No. 4,946,778, to Ladner et al. Single chain antibodies comprise the variable regions of the light and heavy chains joined by a flexible linker moiety. Yet smaller is the antibody fragment known as the single domain antibody or Fd, which comprises an isolated VH single domain. Techniques for obtaining a single domain antibody with at least some of the binding specificity of the full-length antibody from which they are derived are known in the art.

It is possible to determine, without undue experimentation, if an altered or chimeric antibody has the same specificity as the antibody of the HBV#8 antibody, or other HBV antibody, of the invention by ascertaining whether the former blocks the latter from binding to HBV. If the monoclonal antibody being tested competes with the HBV#8 antibody, or other HBV antibody, as shown by a decrease in binding of the HBV#8 antibody, or other HBV antibody, then it is likely that the two monoclonal antibodies bind to the same, or a closely spaced, epitope. Still another way to determine whether a monoclonal antibody has the specificity of the HBV#8 antibody, or other HBV antibody, of the invention is to pre-incubate the HBV#8 antibody, or other HBV antibody, with HBV with which it is normally reactive, and then add the monoclonal antibody being tested to determine if the monoclonal antibody being tested is inhibited in its ability to bind HBV. If the monoclonal antibody being tested is inhibited then, in all likelihood, it has the same, or a functionally equivalent, epitope and specificity as the HBV#8 antibody, or other HBV antibody, of the invention. Screening of monoclonal antibodies of the invention also can be carried out utilizing HBV and determining whether the monoclonal antibody neutralizes HBV.

By using the antibodies of the invention, it is now possible to produce anti-idiotypic antibodies which can be used to screen other monoclonal antibodies to identify whether the antibody has the same binding specificity as an antibody of the invention. In addition, such antiidiotypic antibodies can be used for active immunization (Herlyn, D. et al. 1986 Science 232:100). Such anti-idiotypic antibodies can be produced using well-known hybridoma techniques (Kohler, G. and Milstein, C. 1975 Nature 256:495). An anti-idiotypic antibody is an antibody which recognizes unique determinants present on the monoclonal antibody produced by the cell line of interest. These determinants are located in the hypervariable region of the antibody. It is this region which binds to a given epitope and, thus, is responsible for the specificity of the antibody.

An anti-idiotypic antibody can be prepared by immunizing an animal with the monoclonal antibody of interest. The immunized animal will recognize and respond to the idiotypic determinants of the immunizing antibody and produce an antibody to these idiotypic determinants. By using the anti-idiotypic antibodies of the immunized animal, which are specific for the monoclonal antibodies of the invention, it is possible to identify other clones with the same idiotype as the antibody of the hybridoma used for immunization. Idiotypic identity between monoclonal antibodies of two cell lines demonstrates that the two monoclonal antibodies are the same with respect to their recognition of the same epitopic determinant. Thus, by using anti-idiotypic antibodies, it is possible to identify other hybridomas expressing monoclonal antibodies having the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the image of the epitope bound by the first monoclonal antibody. Thus, the anti-idiotypic monoclonal antibody can be used for immunization, since the anti-idiotype monoclonal antibody binding domain effectively acts as an antigen.

### Nucleic Acids Encoding Anti-HBV Antibodies

Given the disclosure herein of the amino acid sequences of the heavy chain Fd and light chain variable domains of the HBV#8 antibody, or other HBV antibody, one of ordinary skill in the art is now enabled to produce nucleic acids which encode this antibody or which encode the various fragment antibodies or chimeric antibodies described above. It is contemplated that such nucleic acids will be operably joined to other nucleic acids forming a recombinant vector for cloning or for expression of the antibodies of the invention. The present invention includes any recombinant vector containing the coding sequences, or part thereof, whether for prokaryotic or eukaryotic transformation, transfection or gene therapy. Such vectors may be prepared using conventional molecular biology techniques, known to those with skill in the art, and would comprise DNA coding sequences for the immunoglobulin V-regions of the HBV#8 antibody, or other HBV antibody, including framework and CDRs or parts thereof, and a suitable promoter either with (Whittle, N. et al. 1987 Protein Eng. 1:499 and Burton, D. R. et al. 1994 Science 266:1024) or without (Marasco, W. A. et al. 1993 PNAS USA 90:7889 and Duan, L. et al. 1994 PNAS USA 91:5075) a signal sequence for export or secretion. Such vectors may be transformed or transfected into prokaryotic (Huse, W. D. et al. 1989 Science 246:1275; Ward, S. et al. 1989 Nature 341:544; Marks, J. D. et al. 1991 J. Mol. Biol. 222:581; and Barbas, C. F. et al. 1991 PNAS USA 88:7987) or eukaryotic (Whittle, N. et al. 1987 Protein Eng. 1:499 and Burton, D. R. et al. 1994 Science 266:1024) cells or used for gene therapy (Marasco, W. A. et al. 1993 PNAS USA 90:7889 and Duan, L. et al. 1994 PNAS USA 91:5075) by conventional techniques, known to those with skill in the art.

The expression vectors of the present invention include regulatory sequences operably joined to a nucleotide sequence encoding one of the antibodies of the invention. As used herein, the term “regulatory sequences” means nucleotide sequences which are necessary for or conducive to the transcription of a nucleotide sequence ‘which encodes a desired polypeptide and/or which are necessary for or conducive to the translation of the resulting transcript into the desired polypeptide. Regulatory sequences include, but are not limited to, 5′ sequences such as operators, promoters and ribosome binding sequences, and 3′ sequences such as polyadenylation signals. The vectors of the invention may optionally include 5′ leader or signal sequences, 5′ or 3′ sequences encoding fusion products to aid in protein purification, and various markers which aid in the identification or selection of transformants. The choice and design of an appropriate vector is within the ability and discretion of one of ordinary skill in the art. The subsequent purification of the antibodies may be accomplished by any of a variety of standard means known in the art.

A preferred vector for screening monoclonal antibodies, but not necessarily preferred for the mass production of the antibodies of the invention, is a recombinant DNA molecule containing a nucleotide sequence that codes for and is capable of expressing a fusion polypeptide containing, in the direction of amino- to carboxy-terminus, (1) a prokaryotic secretion signal domain, (2) a polypeptide of the invention, and, optionally, (3) a fusion protein domain. The vector includes DNA regulatory sequences for expressing the fusion polypeptide, preferably prokaryotic, regulatory sequences. Such vectors can be constructed by those with skill in the art and have been described by Smith, G. P. et al. (1985 Science 228:1315); Clackson, T. et al. (1991 Nature 352:624); Kang et al. (1991 in “Methods: A Companion to Methods in Enzymology: Vol. 2”; R. A. Lemer and D. R. Burton, ed. Academic Press, NY, pp 111-118); Barbas, C. F. et al. (1991 PNAS USA 88:7978), Roberts, B. L. et al. (1992 PNAS USA 89:2429).

A fusion polypeptide may be useful for purification of the antibodies of the invention. The fusion domain may, for example, include a poly-His tail which allows for purification on Ni+ columns or the maltose binding protein of the commercially available vector pMAL (New England BioLabs, Beverly, Mass.). A currently preferred, but by no means necessary, fusion domain is a filamentous phage membrane anchor. This domain is particularly useful for screening phage display libraries of monoclonal antibodies but may be of less utility for the mass production of antibodies. The filamentous phage membrane anchor is preferably a domain of the cpIII or cpVIII coat protein capable of associating with the matrix of a filamentous phage particle, thereby incorporating the fusion polypeptide onto the phage surface, to enable solid phase binding to specific antigens or epitopes and thereby allow enrichment and selection of the specific antibodies or fragments encoded by the phagemid vector.

The secretion signal is a leader peptide domain of a protein that targets the protein to the membrane of the host cell, such as the periplasmic membrane of Gram-negative bacteria. A preferred secretion signal for E. coli is a pelB secretion signal. The leader sequence of the pelB protein has previously been used as a secretion signal for fusion proteins (Better, M. et al. 1988 Science 240:1041; Sastry, L. et al. 1989 PNAS USA 86:5728; and Mullinax, R. L. et al. 1990 PNAS USA 87:8095). Amino acid residue sequences for other secretion signal polypeptide domains from E. coli useful in this invention can be found in Neidhard, F. C. (ed.), 1987 Escherichia coli and Salmonella Typhimurium: Typhimurium Cellular and Molecular Biology, American Society for Microbiology, Washington, D.C.

To achieve high levels of gene expression in E. coli, it is necessary to use not only strong promoters to generate large quantities of mRNA, but also ribosome binding sites to ensure that the mRNA is efficiently translated. In E. coli, the ribosome binding site includes an initiation codon (AUG) and a sequence 3-9 nucleotides long, located 3-11 nucleotides upstream from the initiation codon (Shine et al. 1975 Nature 254:34). The sequence, which is called the Shine-Dalgarno (SD) sequence, is complementary to the 3′ end of E. coli 16S rRNA. Binding of the ribosome to mRNA and the sequence at the 3′ end of the mRNA can be affected by several factors: the degree of complementarity between the SD sequence and 3′ end of the 16S rRNA; the spacing lying between the SD sequence and the AUG; and the nucleotide sequence following the AUG, which affects ribosome binding. The 3′ regulatory sequences define at least one termination (stop) codon in frame with and operably joined to the heterologous fusion polypeptide.

In preferred embodiments with a prokaryotic expression host, the vector utilized includes a prokaryotic origin of replication or replicon, i.e., a DNA sequence having the ability to direct autonomous replication and maintenance of the recombinant DNA molecule extrachromosomally in a prokaryotic host cell, such as a bacterial host cell, transformed therewith. Such origins of replication are well known in the art. Preferred origins of replication are those that are efficient in the host organism. A preferred host cell is E. coli. For use of a vector in E. coli, a preferred origin of replication is ColEI found in pBR322 and a variety of other common plasmids. Also preferred is the p15A origin of replication found on pACYC and its derivatives. The ColEI and p15A replicons have been extensively utilized in molecular biology, are available on a variety of plasmids and are described by Sambrook. et al., 1989, Molecular Cloning: A Laboratory Manual, 2nd edition, Cold Spring Harbor Laboratory Press.

In addition, those embodiments that include a prokaryotic replicon preferably also include a gene whose expression confers a selective advantage, such as drug resistance, to a bacterial host transformed therewith. Typical bacterial drug resistance genes are those that confer resistance to ampicillin, tetracycline, neomycin/kanamycin or chloramphenicol. Vectors typically also contain convenient restriction sites for insertion of translatable DNA sequences. Exemplary vectors are the plasmids pUC18 and pUC19 and derived vectors such as those commercially available from suppliers such as Invitrogen, (San Diego, Calif.).

When the antibodies of the invention include both heavy chain and light chain sequences, these sequences may be encoded on separate vectors or, more conveniently, may be expressed by a single vector. The heavy and light chain may, after translation or after secretion, form the heterodimeric structure of natural antibody molecules. Such a heterodimeric antibody may or may not be stabilized by disulfide bonds between the heavy and light chains.

A vector for expression of heterodimeric antibodies, such as the full-length antibodies of the invention or the F(ab′)₂, Fab or Fv fragment antibodies of the invention, is a recombinant DNA molecule adapted for receiving and expressing translatable first and second DNA sequences. That is, a DNA expression vector for expressing a heterodimeric antibody provides a system for independently cloning (inserting) the two translatable DNA sequences into two separate cassettes present in the vector, to form two separate cistrons for expressing the first and second polypeptides of a heterodimeric antibody. The DNA expression vector for expressing two cistrons is referred to as a di-cistronic expression vector.

Preferably, the vector comprises a first cassette that includes upstream and downstream DNA regulatory sequences operably joined via a sequence of nucleotides adapted for directional ligation to an insert DNA. The upstream translatable sequence preferably encodes the secretion signal as described above. The cassette includes DNA regulatory sequences for expressing the first antibody polypeptide that is produced when an insert translatable DNA sequence (insert DNA) is directionally inserted into the cassette via the sequence of nucleotides adapted for directional ligation.

The dicistronic expression vector also contains a second cassette for expressing the second antibody polypeptide. The second cassette includes a second translatable DNA sequence that preferably encodes a secretion signal, as described above, operably joined at its 3′ terminus via a sequence of nucleotides adapted for directional ligation to a downstream DNA sequence of the vector that typically defines at least one stop codon in the reading frame of the cassette. The second translatable DNA sequence is operably joined at its 5′ terminus to DNA regulatory sequences forming the 5′ elements. The second cassette is capable, upon insertion of a translatable DNA sequence (insert DNA), of expressing the second fusion polypeptide comprising a secretion signal with a polypeptide coded by the insert DNA.

The antibodies of the present invention may additionally, of course, be produced by eukaryotic cells such as CHO cells, human or mouse hybridomas, immortalized B-lymphoblastoid cells, and the like. In this case, a vector is constructed in which eukaryotic regulatory sequences are operably joined to the nucleotide sequences encoding the antibody polypeptide or polypeptides. The design and selection of an appropriate eukaryotic vector is within the ability and discretion of one of ordinary skill in the art. The subsequent purification of the antibodies may be accomplished by any of a variety of standard means known in the art.

The antibodies of the present invention may furthermore, of course, be produced in plants. In 1989, Hiatt et al. (1989 Nature 342:76) first demonstrated that functional antibodies could be produced in transgenic plants. Since then, a considerable amount of effort has been invested in developing plants for antibody (or “plantibody”) production (for reviews see Giddings, G. et al. 2000 Nat. Biotechnol. 18:1151; Fischer, R. and Emans, N. 2000 Transgenic Res. 9:279). Recombinant antibodies can be targeted to seeds, tubers, or fruits, making administration of antibodies in such plant tissues advantageous for immunization programs in developing countries and worldwide.

In another embodiment, the present invention provides host cells, both prokaryotic and eukaryotic, transformed or transfected with, and therefore including, the vectors of the present invention.

### Diagnostic and Pharmaceutical Anti-HBV Antibody Preparations

The invention also relates to a method for preparing diagnostic or pharmaceutical compositions comprising the monoclonal antibodies of the invention or polynucleotide sequences encoding the antibodies of the invention or part thereof, the pharmaceutical compositions being used for immunoprophylaxis or immunotherapy of HBV disease. The pharmaceutical preparation includes a pharmaceutically acceptable carrier. Such carriers, as used herein, means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

A preferred embodiment of the invention relates to monoclonal antibodies whose heavy chains comprise in CDR3 the polypeptide having SEQ ID NO: 7, and/or whose light chains comprise in CDR3 the polypeptide having SEQ ID NO: 15; and conservative variations of these peptides. Also encompassed by the present invention are certain amino acid sequences that bind to epitopic sequences in hepatitis B surface antigen (HBsAg) and that confer neutralization of HBV when bound thereto. The term “conservative variation” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. The term “conservative variation” also includes the use of a substituted amino acid in place of an unsubstituted parent amino acid provided that antibodies having the substituted polypeptide also bind or neutralize HBV. Analogously, another preferred embodiment of the invention relates to polynucleotides which encode the above noted heavy chain polypeptides and to polynucleotide sequences which are complementary to these polynucleotide sequences. Complementary polynucleotide sequences include those sequences that hybridize to the polynucleotide sequences of the invention under stringent hybridization conditions.

The anti-HBV antibodies of the invention may be labeled by a variety of means for use in diagnostic and/or pharmaceutical applications. There are many different labels and methods of labeling known to those of ordinary skill in the art. Examples of the types of labels which can be used in the present invention include enzymes, radioisotopes, fluorescent compounds, colloidal metals, chemiluminescent compounds, and bioluminescent compounds. Those of ordinary skill in the art will know of other suitable labels for binding to the monoclonal antibodies of the invention, or will be able to ascertain such, using routine experimentation. Furthermore, the binding of these labels to the monoclonal antibodies of the invention can be done using standard techniques common to those of ordinary skill in the art.

Another labeling technique which may result in greater sensitivity consists of coupling the antibodies to low molecular weight haptens. These haptens can then be specifically altered by means of a second reaction. For example, it is common to use haptens such as biotin, which reacts with avidin, or dinitrophenol, pyridoxal, or fluorescein, which can react with specific antihapten antibodies.

The materials for use in the assay of the invention are ideally suited for the preparation of a kit. Such a kit may comprise a carrier means being compartmentalized to receive in close confinement one or more container means such as vials, tubes, and the like, each of the container means comprising one of the separate elements to be used in the method. For example, one of the container means may comprise a monoclonal antibody of the invention that is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label.

In vitro Detection and Diagnostics

The monoclonal antibodies of the invention are suited for in vitro use, for example, in immunoassays in which they can be utilized in liquid phase or bound to a solid phase carrier. In addition, the monoclonal antibodies in these immunoassays can be detectably labeled in various ways. Examples of types of immunoassays which can utilize the monoclonal antibodies of the invention are competitive and non-competitive immunoassays in either a direct or indirect format. Examples of such immunoassays are the radioimmunoassay (RIA) and the sandwich (immunometric) assay. Detection of antigens using the monoclonal antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

The monoclonal antibodies of the invention can be bound to many different carriers and used to detect the presence of HBV. Examples of well-known carriers include glass, polystyrene, polypropylene, polyethylene, dextran, nylon, amylase, natural and modified cellulose, polyacrylamide, agarose and magnetite. The nature of the carrier can be either soluble or insoluble for purposes of the invention. Those skilled in the art will know of other suitable carriers for binding monoclonal antibodies, or will be able to ascertain such, using routine experimentation.

For purposes of the invention, HBV may be detected by the monoclonal antibodies of the invention when present in biological fluids and tissues. Any sample containing a detectable amount of HBV can be used. A sample can be a liquid such as urine, saliva, cerebrospinal fluid, blood, serum or the like; a solid or semi-solid such as tissues, feces, or the like; or, alternatively, a solid tissue such as those commonly used in histological diagnosis.

In vivo Detection of HBV

In using the monoclonal antibodies of the invention for the in vivo detection of antigen, the detectably labeled monoclonal antibody is given in a dose which is diagnostically effective. The term “diagnostically effective” means that the amount of detectably labeled monoclonal antibody is administered in sufficient quantity to enable detection of the site having the HBV antigen for which the monoclonal antibodies are specific.

The concentration of detectably labeled monoclonal antibody which is administered should be sufficient such that the binding to HBV is detectable compared to the background. Further, it is desirable that the detectably labeled monoclonal antibody be rapidly cleared from the circulatory system in order to give the best target-to-background signal ratio.

As a rule, the dosage of detectably labeled monoclonal antibody for in vivo diagnosis will vary depending on such factors as age, sex, and extent of disease of the individual. The dosage of monoclonal antibody can vary from about 0.01 mg/kg to about 50 mg/kg, preferably 0.1 mg/kg to about 20 mg/kg, most preferably about 0.1 mg/kg to about 5 mg/kg. Such dosages may vary, for example, depending on whether multiple injections are given, on the tissue being assayed, and other factors known to those of skill in the art.

For in vivo diagnostic imaging, the type of detection instrument available is a major factor in selecting an appropriate radioisotope. The radioisotope chosen must have a type of decay which is detectable for the given type of instrument. Still another important factor in selecting a radioisotope for in vivo diagnosis is that the half-life of the radioisotope be long enough such that it is still detectable at the time of maximum uptake by the target, but short enough such that deleterious radiation with respect to the host is acceptable. Ideally, a radioisotope used for in vivo imaging will lack a particle emission but produce a large number of photons in the 140-250 keV range, which may be readily detected by conventional gamma cameras.

For in vivo diagnosis, radioisotopes may be bound to immunoglobulin either directly or indirectly by using an intermediate functional group. Intermediate functional groups which often are used to bind radioisotopes which exist as metallic ions are the bifunctional chelating agents such as diethylenetriaminepentacetic acid (DTPA) and ethylenediaminetetra-acetic acid (EDTA) and similar molecules. Typical examples of metallic ions which can be bound to the monoclonal antibodies of the invention are ¹¹¹In, ⁹⁷Ru, ⁶⁷Ga, ⁶⁸Ga, ⁷²As, ⁸⁹Zr and ²⁰¹Tl.

The monoclonal antibodies of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements which are particularly useful in such techniques include ¹⁵⁷Gd, ⁵⁵Mn, ¹⁶²Dy, ⁵²Cr and ⁵⁶Fe.

The monoclonal antibodies of the invention can be used in vitro and in vivo to monitor the course of HBV disease therapy. Thus, for example, by measuring the increase or decrease in the number of cells infected with HBV or changes in the concentration of HBV present in the body or in various body fluids, it would be possible to determine whether a particular therapeutic regimen aimed at ameliorating HBV disease is effective.

### Prophylaxis and Therapy of HBV Disease

The monoclonal antibodies can also be used in prophylaxis and as therapy for HBV disease in humans. The terms, “prophylaxis” and “therapy” as used herein in conjunction with the monoclonal antibodies of the invention denote both prophylactic as well as therapeutic administration and both passive immunization with substantially purified polypeptide products, as well as gene therapy by transfer of polynucleotide sequences encoding the product or part thereof. Thus, the monoclonal antibodies can be administered to high-risk subjects in order to lessen the likelihood and/or severity of HBV disease or administered to subjects already evidencing active HBV infection. In the present invention, Fab fragments also bind or neutralize HBV and therefore may be used to treat HBV infection but full-length antibody molecules are otherwise preferred.

As used herein, a “prophylactically effective amount” of the monoclonal antibodies of the invention is a dosage large enough to produce the desired effect in the protection of individuals against HBV virus infection for a reasonable period of time, such as one to two months or longer following administration. A prophylactically effective amount is not, however, a dosage so large as to cause adverse side effects, such as hyperviscosity syndromes, pulmonary edema, congestive heart failure, and the like. Generally, a prophylactically effective amount may vary with the subject's age, condition, and sex, as well as the extent of the disease in the subject and can be determined by one of skill in the art. The dosage of the prophylactically effective amount may be adjusted by the individual physician or veterinarian in the event of any complication. A prophylactically effective amount may vary from about 0.01 mg/kg to about 50 mg/kg, preferably from about 0.1 mg/kg to about 20 mg/kg, most preferably from about 0.2 mg/kg to about 5 mg/kg, in one or more administrations (priming and boosting).

As used herein, a “therapeutically effective amount” of the monoclonal antibodies of the invention is a dosage large enough to produce the desired effect in which the symptoms of HBV disease are ameliorated or the likelihood of infection is decreased. A therapeutically effective amount is not, however, a dosage so large as to cause adverse side effects, such as hyperviscosity syndromes, pulmonary edema, congestive heart failure, and the like. Generally, a therapeutically effective amount may vary with the subject's age, condition, and sex, as well as the extent of the disease in the subject and can be determined by one of skill in the art. The dosage of the therapeutically effective amount may be adjusted by the individual physician or veterinarian in the event of any complication. A therapeutically effective amount may vary from about 0.01 mg/kg to about 50 mg/kg, preferably from about 0.1 mg/kg to about 20 mg/kg, most preferably from about 0.2 mg/kg to about 5 mg/kg, in one or more dose administrations daily, for one or several days. Preferred is administration of the antibody for 2 to 5 or more consecutive days in order to avoid “rebound” of virus replication from occurring.

The monoclonal antibodies of the invention can be administered by injection or by gradual infusion over time. The administration of the monoclonal antibodies of the invention may, for example, be intravenous, intraperitoneal, intramuscular, intracavity, subcutaneous, or transdermal. Techniques for preparing injectate or infusate delivery systems containing antibodies are well known to those of skill in the art. Generally, such systems should utilize components which will not significantly impair the biological properties of the antibodies, such as the paratope binding capacity (see, for example, Remington's Pharmaceutical Sciences, 18th edition, 1990, Mack Publishing). Those of skill in the art can readily determine the various parameters and conditions for producing antibody injectates or infusates without resort to undue experimentation.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and the like.

### Isolation By Phage Display And Characterization Of A Chimpanzee Monoclonal Antibody That Neutralizes Hepatitis B Virus

Isolation of HBsAg-specific Fabs. Chimpanzee 1441 had been experimentally infected with HBV strain MS-2 (ayw). Prior to the bone marrow sampling, the chimpanzee was immunized once with the HBV vaccine, Engerix-B (adw), in order to raise its serum IgG titers to HBsAg. RNA was extracted from bone marrow lymphocytes. Messenger RNA was reverse transcribed using an oligo-dT primer to generate cDNA. Human IgG1-specific primers were then used to amplify both the κ-chain and γ1-chain antibody genes using PCR (see Example 1 for details). These products were purified and cloned into the phage display vector, pComb3H. The resultant Fab-phage library was then panned against HBsAg. The a determinant of HBsAg is the principle determinant to which neutralizing antibodies are directed. In order to increase the likelihood of generating a determinant-specific antibodies, a heterologous subtype of HBsAg (adw) was used as the panning antigen. After four rounds of panning, the DNA from the enriched phage library was isolated and modified by restriction enzyme digestion to allow soluble Fab expression in E. coli. A total of fifty clones were analyzed, of which seventeen were determined to be HBsAg-specific after the initial screening. A subsequent ELISA confirmed all seventeen Fabs as HBsAg-specific when reactivity was compared to a panel of unrelated proteins.

Sequence analysis of HBsAg-specific Fabs. Restriction digestion analysis of the seventeen HBsAg-specific Fabs was carried out using Bst NI. This restriction enzyme cuts frequently in the γ1-heavy chain. Only one digestion pattern was observed, suggesting all the clones had the same heavy chain sequence. Ten clones were sequenced, and one unique γ1-heavy chain was identified. This was represented by clone HBV#8 (FIG. 1).

We attempted to determine the specific germ-line origin of HBV#8 by conducting a sequence similarity search of all the known human immunoglobulin genes. The findings are summarized in Table 1. The nucleotide sequence of the γ1-heavy chain exhibited the most homology with the human VH4 family of germ line segments. Specifically, HBV#8 was most closely related to 3d279d, with 86% homology at the nucleotide level for the whole VH region. The homology increased to 88% when the complementarity determining regions 1 and 2 (CDR1 and CDR2) were excluded. The CDRs are loops of amino acids that determine antigen specificity. Therefore, they will be different for each antibody. The intervening framework regions of the antibody are relatively invariant, hence the increase in the level of homology. The κ-light chain sequence exhibited the most homology with the human Via family of germ line segments. HBV#8 κ-chain shared 90% nucleotide sequence homology with HK137), for the whole Vκ region, and 91% excluding CDR1 and CDR2.

Identification of HBV#8 subtype specificity. To determine the HBV subtype specificity, an antigen capture ELISA was performed. Dilutions of HBV#8 Fab were bound to Ni²⁺-coated wells via a histidine tag at the end of the Fab CH1 domain. Plasma samples from chimpanzees infected with four of the most common HBV subtypes (adw, ayw, adr, and ayr) were incubated in the Fab coated wells. Captured HBsAg was detected with serum from chimpanzee 1441 (taken at the time of bone marrow sampling) and an anti-human IgG (Fc specific) secondary antibody. HBV#8 Fab captured antigen from all four of the HBV subtypes tested. Therefore, both chimpanzee 1441 and HBV#8 recognized a universal epitope, possibly the a determinant, on HBsAg. HBV#8 Fab also captured antigen from two chimpanzee plasma samples containing an HBV vaccine escape mutant virus. Therefore, the amino acid mutation at codon 145, which characterizes this vaccine escape mutant, does not form a critical part of the epitope recognized by HBV#8.

HBV#8 affinity determination. The affinities of both the Fab and IgG forms of HBV#8 for HBsAg were determined by competition inhibition ELISA. The concentration of free HBsAg required to inhibit antibody binding by 50% is equivalent to the equilibrium dissociation constant (K_{d}). The K_{d} value of HBV#8 was 1.9×10⁻⁶ M for the Fab, and 2.5×10⁻⁸M for the IgG. Therefore, bivalency improves HBV#8 affinity by 75-fold.

Neutralization of HBV by Fab HBV#8. The ability of HBV#8 Fab to neutralize HBV in vitro was determined in the primary hepatocyte cell culture system described by Gripon, P. et al. (1988 J. Virol. 62:4136-4143) and Gripon, P. et al. (1993 Virology 192:534-540). HBV was incubated with log₁₀ dilutions of HBV#8 Fab prior to inoculation onto the primary hepatocyte monolayers. After 12 days of culture, HBV replication was determined by (1) the amount of HBsAg in the cell supernatants using RIA; and (2) the amount of HBV DNA in cell lysates using Southern blot. A known HBV neutralizing MAb, CS131A, was used as a positive control. HBV#8 Fab neutralized HBV at the highest concentration tested, 10 μg ml⁻¹, in two separate neutralization tests. The levels of HBsAg in cell supernatants were reduced by >90% of the control (Table 2), and levels of HBV DNA in the cell lysates were diminished. MAb CS131A neutralized the inoculum at 0.1 μg ml⁻¹, a 100-fold lower concentration than HBV#8 Fab (Table 2).

Epitope mapping. An indirect competition assay was performed to identify the location of the epitope recognized by HBV#8 compared to the locations of the epitopes identified by a panel of mouse MAbs whose epitopes had been mapped previously. MAbs H35, H5, H53, H166, RFHBs1, RFHBs2, RFHBs4, RFHBs7, RFHBs15, and RFHBs16 were incubated with HBsAg-coated wells and the subsequent binding of HBV#8 was determined. MAb H53 blocked the binding of biotinylated HBV#8 Fab, and of HBV#8 IgG to HBsAg (Table 3). MAb RFHBs16 also consistently reduced binding of HBV#8 Fab and IgG by approximately 50%, suggesting that the two epitopes are in close proximity to each other on the HBsAg molecule, although not overlapping or immediately adjacent. In addition, MAbs H166 and RFHBs1 inhibited HBV #8 IgG binding. However, as with RFHBs16, inhibition was not complete, suggesting that these epitopes are not immediately adjacent or overlapping. At the highest concentration of unlabeled HBV#8 Fab attainable, HBV#8 Fab (biotinylated) binding was inhibited by 66%, and HBV#8 IgG binding was inhibited by 57%.

### EXAMPLE 1

Donor animal. A bone marrow aspirate was taken from the pelvis of chimpanzee (Pan troglodytes) 1441. This animal had been experimentally infected with HAV, HBV, HCV, HDV and HEV previously. Prior to the aspirate being taken, the animal was boosted with a commercial HAV vaccine (HAVRIX, SmithKline Beecham), the HBV vaccine (Engerix-B, SmithKline Beecham), and purified baculovirus-expressed HEV ORF2 protein. The bone marrow cells were stored as a viable single cell suspension in 10% dimethyl sulfoxide, 10% fetal calf serum and RPMI 1640 medium (BioWhittaker) in liquid nitrogen.

Construction of γ1/κ antibody gene library. Total RNA was extracted from an aliquot of bone marrow cells (RNA Isolation Kit; Stratagene) and mRNA was reverse transcribed into cDNA using an oligo dT primer (First Strand Synthesis Kit, Gibco/BRL). The cDNAs were amplified by PCR using rTth DNA polymerase (Perkin Elmer). Thirty cycles of 94° C. for 15 s, 52° C. for 50 s, and 68° C. for 90 s were performed. Chimpanzee κ-chain genes were amplified using primers specific for the human κ-chain genes. Fd segments (variable and first constant domains) of the chimpanzee γ1-chain genes were amplified with nine family-specific human VH primers recognizing the 5′ end of the genes (Persson, M. A. et al. 1991 PNAS USA 88:2432-2436; Barbas, C. F. 3rd et al. 1991 PNAS USA 88:7978-7982) and a chimpanzee γ1-specific 3′ primer (5′-GCATGTACTAGTTGTGTCACAAGATTTGGG-3′) (SEQ ID NO: 17) (3′ primer sequence determined from Vijh-Warrier, S. et al. 1995 Mol. Immunol. 32:1081-1092).

The library construction using the pComb3H surface display vector was carried out as described by Glamann, J. et al. (1998 J. Virol. 72:585-592) and Williamson, R. A. et al. (1993 PNAS USA. 90:4141-4145). The final library of 1.9×10⁷ clones was stored in 12.5% glycerol-LB broth at −80° C. until use.

Panning and ELISA reagents. In all panning experiments and enzyme-linked immunosorbant assays (ELISA) HBsAg, purified from human plasma, was diluted to 1.0 μg ml⁻¹ in 50 mM sodium carbonate buffer (pH 9.6), and coated on to EIA/RIA A/2 plates (Costar) overnight at 4° C. A goat anti-human IgG (H+L)-specific antibody (Pierce) was used to detect Fab production, and this was coated to microtiter wells at a dilution of 1:1000, in 50 mM sodium carbonate buffer (pH 9.6), as above.

Library Screening. Screening of the combinatorial library was carried out according to the method described by Barbas, C. F. 3rd et al. (1991 PNAS USA 88:7978-7982) and Williamson, R. A. et al. (1993 PNAS USA. 90:4141-4145). Approximately 10⁹ bacteria from the library stock were grown up and infected with helper phage, VCS M13 (Stratagene), added at a multiplicity of infection of 50, to produce the library displayed on the surface of phage particles. Phage were panned on ELISA wells coated with HBsAg in all, four rounds of panning were performed. After amplification of the selected library, the phagemid DNA was extracted and soluble Fabs produced by restriction enzyme digestion of the phagemid vector to remove the bacteriophage coat protein III-encoding region of the phage (Bender, E. et al. 1993 Hum. Antibodies Hybridomas. 4:74-79). The phagemid DNAs were religated and transformed into Escherichia coli XL-1 Blue (Stratagene). A total of 50 colonies were picked and each inoculated into Luria-Bertani broth (Gibco/BRL) supplemented with 100 μg ml⁻¹ ampicillin and 1% (v/v) glucose (Sigma) in a single well of a 96-well microtiter plate. Bacteria were incubated at 30° C. overnight and Fab production induced according to Glamann, J. et al. (1998 J. Virol. 72:585-592). The bacterial supernatants were tested by ELISA for reactivity with HBsAg and for Fab production.

Fab production, purification and biotinylation. Fab purification was facilitated by modification of the vector, pCOMB3H, to encode a six-histidine tail at the end of the soluble Fab fragment (modification carried out by, and detailed in Glamann, J. et al. (1998 J. Virol. 72:585-592).

Bacterial culture and Fab fragment purification were carried out as described by (Glamann, J. et al. 1998 J Virol. 72:585-592). Protein concentrations were determined by both dye binding assay (Bio-Rad) and A280 nm (using the extinction co-efficient of 1.4 optical density units equivalent to 1.0 mg ml⁻¹). The Fab purity was determined by polyacrylamide gel electrophoresis with colloidal coomassie blue staining (Sigma).

The purified Fabs were diluted in sodium bicarbonate buffer (pH 9.0), and biotinylated at 4° C. as per the manufacturer's protocol (Pierce). After biotinylation, the Fabs were dialysed against PBS overnight at 4° C., and concentrated in Centricon-30 concentrators (Amicon) as required.

ELISA analysis of Fab specificity. Protein antigens used in ELISA were coated onto microtiter plates overnight at 4° C. using the following concentrations: HBsAg protein at 1.0 μg ml⁻¹, thyroglobulin, lysozyme, glyceraldehyde-3-phopshate, chicken albumin and cytochrome C (Sigma) at 10.0 μg ml⁻¹. Antigen-coated wells were blocked for 1 h at room temperature with 3% bovine serum albumin (BSA)-phosphate buffered saline (PBS), washed twice with PBS -Tween 20 (0.05% (v/v)), and 50 μl of crude or purified Fab added to the wells. After 1 h incubation at 37° C., plates were washed six times with PBS-Tween 20. Bound Fab were detected with 1:1500 dilution of a goat anti-human F(ab′)₂ alkaline phosphatase labeled secondary antibody (Pierce). The color was developed with 1 mg ml⁻¹ p-nitrophenyl phosphate (Sigma) in diethanolamine buffer (Pierce) as the substrate. Optical density was determined at 405 nm with a reference wavelength of 650 nm.

Restriction digestion pattern analysis and nucleic acid sequence analysis of HBV-specific Fab clones. For Bst N1 (New England Biologicals) fingerprinting, 1 μg of plasmid DNA was digested with 1 U of enzyme overnight at 60° C. The restriction patterns were analyzed on a 3% agarose gel. Nucleic acid sequencing was carried out with the ABI PRISM Dye Terminator Cycle Sequencing Ready Reaction kit by using Ampli-Taq DNA Polymerase (Perkin-Elmer) and the following sequencing primers: heavy chain, 5′-ATTGCCTACGGCAGCCGCTGG-3′ (HC1) (SEQ ID NO: 18) and 5′-GGAAGTAGTCCTTGACCAGGC-3′ (HC4) (SEQ ID NO: 19); κ chain, 5′-ACAGCTATCGCGATTGCAGTG-3′ (LC1) (SEQ ID NO: 20) and 5′-CACCTGATCCTCAGATGGCGG-3′ (LC4) (SEQ ID NO: 21) (Glamann, J. et al. 1998 J Virol. 72:585-592). The sequences were analyzed using the GeneWorks (Oxford Molecular Group) software package. Sequence similarity searches were performed using the V-BASE program, which is a compilation of all available human variable segment Ig germ line sequences (Cook, G. P. and Tomlinson I. M. 1995 Immunol. Today. 16:237-242).

Generation of HBV#8 IgG and expression in COS-7 cells. The HBV#8 phage display vector was digested with the restriction enzymes XbaI and SstI, or XhoI and AgeI for sub-cloning of the κ-light chain and γ1-heavy chain inserts into the whole IgG mammalian expression vectors, pCNLC and pCDHC respectively (Trill, J. J. et al. 1995 Curr. Opin. Biotechnol. 6:553-560). The whole IgG vectors were then co-transfected into COS-7 cells using a cationic lipid reagent (Superfect, Qiagen), according to the method of Ames, R. S. et al. (1995 J Immunol. Methods 184:177-186). The cells were incubated for 21 days at 37° C. in a CO₂ incubator. Cell supernatants were harvested at 7 day intervals, and tested for HBsAg-specific IgG production by ELISA using an anti-human IgG (Fe-specific) alkaline phosphatase secondary antibody (Sigma).

HBV subtyping ELISA. The HBV#8 Fab subtype specificity was determined by antigen capture ELISA. Dilutions of HBV#8 were incubated on Ni²⁺ coated wells (Pierce) for 1 h at ₃₇° _{C.;} the plate was washed four times with PBS/Tween-20 (0.05%), and blocked with 3% BSA/PBS for 1 h at room temperature. Plasma samples from chimpanzees infected with the different HBV subtypes (ayr, adr, adw, and ayw) were diluted 1:10 in 3% BSA/PBS and incubated in the wells for 1 h 30 min. at 37° C. After 6 washes, a 1:750 dilution of Chimpanzee 1441 serum was added to the wells and incubated for 1 h at 37° C. The plate was washed four times, and captured HBV detected by addition of a 1:5000 dilution of anti-human IgG (Fc specific) alkaline phosphatase-labeled secondary antibody (Pierce). The color was developed as described above.

Competition ELISA for affinity determination. The affinity (equilibrium dissociation constant, Kd) of HBV#8 Fab and IgG was determined by competition inhibition ELISA (Rath, S. et al. 1988 J. Immunol. Methods 106:245-249; Persson, M. A. A. et al. 1991 PNAS USA 88:2432-2436). Briefly, log10 dilutions of HBV#8 were titrated on HBsAg (Biodesign) coated wells, and the dilution at which 10-fold decrease in Fab concentration gave a substantial reduction in the binding of the MAb was used in the competition ELISA. This concentration of HBV#8 was then incubated for 2 h at 37° C. with decreasing log10 concentrations of HBsAg in solution, in HBsAg-coated wells. The plates were washed four times with PBS/Tween-20, and bound Fab was detected using anti-human IgG (Fab-specific) alkaline phosphatase-labeled secondary antibody (Sigma). The percent reduction in A405 nm value was plotted and the 50% inhibition (I₅₀) value was extrapolated. The I₅₀ concentration of antigen was then multiplied by a factor of 100 to give the Kd affinity value since our HBsAg was derived from purified 22 nm particles containing approximately 100 copies of the monomer per particle.

In vitro neutralization Assay. The in vitro neutralization assay was performed as described by Gripon, P. et al. (1988 J. Viral. 62:4136-4143) and Gripon, P. et al. (1993 Virology 192:534-540). Briefly, the HBV inocula were incubated with log₁₀ dilutions of HBV#8 Fab or MAb CS131A for 1 h at room temperature. The virus-antibody mixtures were then incubated overnight at 37° C. on human hepatocyte cultures. Following extensive washing with maintenance medium, the hepatocytes were maintained for 12 days at 37° C. On day 12 the supernatants were harvested and analyzed for the presence of HBsAg by radioimmunoassay. The hepatocytes were analyzed for the presence of intracellular viral DNA by southern blot.

Epitope mapping by indirect competition ELISA. Competing MAbs were titrated on HBsAg-coated wells and the dilution determined which gave an A_{405 nm} reading of approximately 1.0, but at a concentration that did not saturate the antigen coated to the plate. Dilutions of all the MAbs were incubated on HBsAg-coated wells for 1 h at 37° C., washed four times, and then a single dilution of the competing MAb was incubated in all wells for 1 h at 37° C. Binding of the competitor MAb was detected using either an anti-mouse IgG (H+L chain specific) alkaline phosphatase-conjugated antibody (Pierce), anti-human IgG (Fc specific) alkaline phosphatase-conjugated antibody (Sigma), or strepavidin-alkaline phosphatase (Pierce). The color was developed as described above.

TABLE 1

| MAb | V_{H} | D | J_{H} | V_{K} | I. J_{K} |
|---|---|---|---|---|---|
| HBV#8 | 3d279d | ND | JH6c | HK137 | JK1 |

ND-not determined due to lack of identifiable homolog

TABLE 2

| | II. CS131A | | III. HBV#8 FAB | |
|---|---|---|---|---|
| MAb (μg ml⁻¹) | P/N^{⊥} | % | P/N | % |
| 10.0 | 6.2 | 6 | 4.1 | 4 |
| 1.0 | 6.3 | 6 | 55.8 | 59 |
| 0.1 | 8.4 | 8 | 83.3 | 88 |
| 0.01 | 75.7 | 73 | 82 | 86 |
| 0.0 | 103 | 100 | 94.9 | 100 |
| Cell control | 1.6 | 2 | 1.85 | 2 |

^{⊥}P/N ratio of >2.1 is considered positive

TABLE 3

| | HBV#8 | |
|---|---|---|
| MAb | Fab | IgG |
| H53 | + | + |
| H35 | − | − |
| H5 | − | − |
| H166 | − | ± |
| RFHBs 1 | − | ± |
| RFHBs 2 | − | − |
| RFHBs 4 | − | − |
| RFHBs 7 | − | − |
| RFHBs 15 | − | − |
| RFHBs 16 | ± | ± |
| HBV#8 Fab* | ± | ± |

*starting at 1:3 dilution (remainder at 1:10)
+ = 100-75% inhibition of binding
± = 74-50% inhibition of binding
− = <50% inhibition of binding

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

1. A substantially pure polypeptide comprising
a fully human or humanized chimpanzee monoclonal antibody that binds or neutralizes hepatitis B virus (HBV),
or comprising
a monoclonal antibody that binds the antigen to which monoclonal antibody HBV#8 (ATCC Accession No. PTA-6098) binds.

2. The substantially pure polypeptide of claim 1 wherein said antibody comprises an Fd fragment.

3. The substantially pure polypeptide of claim 1 wherein said antibody comprises an Fab fragment.

4. The substantially pure polypeptide of claim 1 wherein said antibody includes a heavy chain CDR3 region having the amino acid sequence of SEQ ID NO: 7.

5. The substantially pure polypeptide of claim 4 wherein said antibody includes a heavy chain CDR2 region having the amino acid sequence of SEQ ID NO: 5.

6. The substantially pure polypeptide of claim 5 wherein said antibody includes a heavy chain CDR1 region having the amino acid sequence of SEQ ID NO: 3.

7. The substantially pure polypeptide of claim 4 wherein said antibody includes a heavy chain Fd region including the amino acid sequence of SEQ ID NO: 1.

8. The substantially pure polypeptide of claim 4 wherein said antibody includes a light chain CDR3 region having the amino acid sequence of SEQ ID NO: 15.

9. The substantially pure polypeptide of claim 8 wherein said antibody includes a light chain CDR2 region having the amino acid sequence of SEQ ID NO: 13.

10. The substantially pure polypeptide of claim 9 wherein said antibody includes a light chain CDR1 region having the amino acid sequence of SEQ ID NO: 11.

11. The substantially pure polypeptide of claim 4 wherein said antibody includes a light chain region including the amino acid sequence of SEQ ID NO: 9.

12. The substantially pure polypeptide of claim 4 wherein said antibody includes a heavy chain Fd region including the CDR amino acid sequences of SEQ ID NO: 1.

13. The substantially pure polypeptide of claim 12 wherein said antibody includes a light chain region including the CDR amino acid sequences of SEQ ID NO: 9.

14. An isolated nucleic acid comprising
a nucleotide sequence encoding a polypeptide selected from the group consisting of the polypeptide of claim 4.

15. An isolated nucleic acid as in claim 14 wherein
said nucleic acid comprises a vector including a regulatory sequence operably joined to said nucleic acid.

16. A host cell including a vector comprising a nucleic acid of claim 14.

17. A pharmaceutical preparation comprising
a pharmaceutically acceptable carrier; and
a substantially pure polypeptide selected from the group consisting of the polypeptide of claim 4.

18. A diagnostic preparation comprising
a pharmaceutically acceptable carrier; and
a substantially pure polypeptide selected from the group consisting of the polypeptide of claim 4.

19. A method for the treatment of hepatitis B virus (HBV) disease comprising
administering to a patient a therapeutically effective amount of the pharmaceutical preparation of claim 17.

20. A method for prophylaxis against hepatitis B virus (HBV) disease comprising
administering to a patient a prophylactically effective amount of the pharmaceutical preparation of claim 17.

21. A method for the diagnosis of hepatitis B virus (HBV) infection comprising
administering to a patient an effective amount of the diagnostic preparation of claim 18, and
detecting binding of the substantially pure polypeptide as a determination of the presence of hepatitis B virus (HBV) infection.

22. A method of detecting the presence of hepatitis B virus (HBV) in a biological sample comprising
contacting said sample with the diagnostic preparation of claim 18, and
assaying binding of the substantially pure polypeptide as a determination of the presence of said hepatitis B virus (HBV).

23. HBV#8 deposited with ATCC as ATCC Accession No. PTA-6098.
